(19) United States
(12) Patent Application Publication
(10) Pub. No.: US 2024/0018586 A1
(43) Pub. Date: Jan. 18, 2024

(54) HLA HAPLOTYPES AS BIOMARKERS FOR COVID-19 SYMPTOM SEVERITY

(71) Applicant: PXD Limited, Newcastle upon Tyne, Tyne and Wear (GB)

(72) Inventor: David Langton, Newcastle upon Tyne, Tyne and Wear (GB)

(21) Appl. No.: 18/039,631

(22) PCT Filed: Nov. 26, 2021
(86) PCT No.: PCT/GB2021/053084

§ 371 Date: May 31, 2023

(30) Foreign Application Priority Data
Dec. 1, 2020 (GB) 2018922.1

(51) Int. Cl. C12Q 1/6881; G01N 33/82

(52) U.S. Cl. CPC C12Q 1/6881; G01N 33/82; G01N 2333/70539; G01N 2800/50; C12Q 2600/172; G01N 2800/52

(57) ABSTRACT

The present invention relates to methods, devices and kits for identifying individuals who are at increased risk of developing severe symptoms associated with severe acute respiratory syndrome coronavirus 2 (SARS-CoV-2) infection. The present invention also relates to methods, devices and kits for identifying individuals who have a reduced risk of developing severe symptoms associated with SARS-CoV-2 infection. In particular, it relates to the use of HLA haplotypes as biomarkers to predict disease severity in individuals infected with SARS-CoV-2.

The present invention relates to methods, devices and kits for identifying individuals who are at increased risk of developing severe symptoms associated with severe acute respiratory syndrome coronavirus 2 (SARS-CoV-2) infection. The present invention also relates to methods, devices and kits for identifying individuals who have a reduced risk of developing severe symptoms associated with SARS-CoV-2 infection. In particular, it relates to the use of HLA haplotypes as biomarkers to predict disease severity in individuals infected with SARS-CoV-2.

Severe acute respiratory syndrome coronavirus 2 (SARS-CoV-2) is a novel coronavirus which is the causative agent of coronavirus disease (Covid-19) in patients. SARS-CoV-2 was first identified in Hubei province, China in December 2019 as the causative agent of a pneumonia in patients. Since its detection, the virus has spread widely and rapidly. In March 2020, the World Health Organisation (WHO) declared the outbreak of Covid-19 a pandemic, indicating worldwide spread of the disease.

In the months following the initial detection of SARS-CoV-2, according to Johns Hopkins Covid-19 dashboard there have been over 50 million cases of Covid-19 worldwide resulting over 1.25 million deaths (estimates true as of 9 Nov. 2020). The worldwide spread of Covid-19 has also had significant economic consequences globally.

In humans, SARS-CoV-2 infection is causative of Covid-19. Symptoms of Covid-19 vary from patient to patient but most commonly include a persistent cough, anosmia and fever. Symptom severity in Covid-19 varies, with some patients being asymptomatic and others requiring hospitalisation and respiratory support. There are some widely acknowledged factors that increase individual risk for developing a severe disease resulting from SARS-CoV-2 infection, such as age and BMI. However, these factors are not able to accurately predict disease severity in all individuals suggesting other risk factors exist.

Significant resource is being directed towards the development of a vaccine against SARS-CoV-2. Although no SARS-CoV-2 vaccines are yet approved for use in the UK, there are several ongoing clinical trials. In the event that an effective vaccine is approved for use in the UK, it will be important to prioritise vaccination for those most at risk from developing a severe disease from SARS-CoV-2 infection. Whilst it is possible to do so by applying the currently acknowledged risk factors (i.e. age and BMI), this will not protect those who fall outside these categories but are still vulnerable to severe disease. It is therefore desirable to improve the methods by which we determine individual risk for developing severe symptoms associated with SARS-CoV-2 infection.

Furthermore, it is important that healthcare systems are able to predict which patients are at a higher risk of developing a severe disease resulting from SARS-CoV-2 infection. The ability to identify those at greater risk of developing severe disease will enable healthcare professionals to monitor these patients and administer treatments, such as corticosteroids which have proven to lower mortality in patients more closely, early in disease progression.

The human leukocyte antigen (HLA) system is a vital aspect of the immune system. The HLA system, also known as the major histocompatibility complex (MHC) in humans, is a group of cell-surface proteins with a range functions in the immune system. HLA proteins can be split into three classes—HLA class I, HLA class II and HLA class III. HLA class I proteins present peptides from within the cell on the cell surface, allowing cells infected with foreign antigens to be detected and destroyed. HLA class I includes HLA-A, HLA-B and HLA-C proteins, each of which are encoded by genes of the same name. HLA class II proteins present peptides external to the cell on the cell surface, allowing for the production of antigen-specific antibodies. HLA class II proteins include HLA-DP, HLA-DM, HLA-DO, HLA-DQ. and HLA-DR. HLA class III proteins are involved in the complement system.

The HLA genes reside on the short arm of human chromosome 6 and are among the most polymorphic protein-coding genes in the human genome. The HLA genes are closely linked and the entire HLA complex is inherited as a haplotype in a Mendelian manner, one from each parent. Haplotyping the HLA genes is important in many applications, notably in the context of allogenic transplantation where HLA matching is vital to reduce the risk of post-transplantation complications. There are multiple methods of HLA haplotyping, commonly these are PCR-based techniques but increasingly, next-generation sequencing (NGS) is used.

### SUMMARY OF THE INVENTION

According to a first aspect of the present invention, there is provided a method of assessing the risk of an individual developing severe symptoms when infected with SARS-CoV-2, the method comprising: determining whether the individual has at least one of the following increased risk HLA haplotypes selected from DQA1*01:02/DQB1*06:02; DRB1*15:01; DQA1*05:01/DQB1*02:01; HLA-B*44:03; HLA-C*04:01; HLA-A*11:01; DRB1*04:03; DRB1*04:05; DRB1*04:02; and when the individual has at least one of the following increased risk HLA haplotypes selected from: DQA1*01:02/DQB1*06:02; DRB1*15:01; DQA1*05:01/DQB1*02:01; HLA-B*44:03; HLA-C*04:01; HLA-A*11:01; DRB1*04:03; DRB1*04:05; DRB1*04:02; determining that the individual is at increased risk of developing severe symptoms associated with SARS-CoV-2 infection.

According to a second aspect of the present invention, there is provided a method of assessing the risk of an individual developing severe symptoms when infected with SARS-CoV-2, the method comprising: determining whether the individual has at least one of the following increased risk HLA haplotypes selected from: DQA1*01:02/DQB1*06:02; DRB1*15:01; DQA1*05:01/DQB1*02:01; HLA-B*44:03; HLA-C*04:01; HLA-A*11:01; DRB1*04:03; DRB1*04:05; DRB1*04:02; and when the individual has at least one of the following increased risk HLA haplotypes selected from: HLA-B*44:03; HLA-C*04:01; HLA-A*11:01; DRB1*04:03; DRB1*04:05; DRB1*04:02; determining that the individual is at increased risk of developing severe symptoms associated with SARS-CoV-2 infection; and where the individual has at least one of the increased risk HLA haplotypes selected from: DQA1*01:02/DQB1*06:02; DRB1*15:01; DQA1*05:01/DQB1*02:01;carrying out the further step of determining the vitamin D level of the individual, or estimating the vitamin D status of the individual by means of at least one surrogate marker of vitamin D status; and where the individual with at least one of the increased risk HLA haplotypes selected from: DQA1*01:02/DQB1*06:02; DRB1*15:01; DQA1*05:01/DQB1*02:01; also has a vitamin D level below a predetermined threshold, or has a vitamin D status that is estimated to be low based on the at least one surrogate marker, determining that the individual is at increased risk of developing severe symptoms associated with SARS-CoV-2 infection.

According to a third aspect of the present invention, there is provided a method of assessing the risk of an individual developing severe symptoms when infected with SARS-CoV-2, the method comprising: determining whether the individual has at least one of the following increased risk HLA haplotypes selected from: DQA1*01:02/DQB1*06:02; DRB1*15:01; DQA1*05:01/DQB1*02:01; determining the vitamin D level of the individual, or estimating the vitamin D status of the individual by means of at least one surrogate marker of vitamin D status; and determining when the individual has at least one of the following increased risk HLA haplotypes selected from: DQA1*01:02/DQB1*06:02; DRB1*15:01; DQA1*05:01/DQB1*02:01; and has a vitamin D level below a predetermined threshold, or has a vitamin D status that is estimated to be low based on the at least one surrogate marker, the individual is at increased risk of developing severe symptoms associated with SARS-CoV-2 infection.

Advantageously, identifying the presence an increased risk HLA haplotype and low vitamin D levels in an individual indicates that the individual has an increased risk of developing severe symptoms if infected with SARS-CoV-2. This knowledge allows an individual to modify their lifestyle to reduce their risk of being infected, for example by reducing their social contact.

Furthermore, the method provides a means of identifying those at increased risk from developing severe symptoms resulting from SARS-CoV-2 infection which can be used when prioritising individuals for receipt of prophylactic treatments such as vaccinations.

Advantageously, the ability to determine whether an individual has an increased risk of developing severe symptoms associated with SARS-CoV-2 infection allows health care professionals to more closely monitor and/or administer treatment sooner in high risk individuals, improving the likelihood of recovery in such individuals.

Optionally, the method according to the first, second or third aspect of the present invention further comprises the step of determining at least one of the following: individual's age; individual's BMI; individual's gender; wherein the individual is at a greater increased risk of developing severe symptoms associated with SARS-CoV-2 infection if the individual is aged over 40 years old; the individual has a BMI over 25; the individual is male.

Optionally, symptoms comprise one or more selected from: anosmia, headache, fever, general malaise, joint pain, gastrointestinal disturbance, cough, dyspnoea.

Optionally, severe symptoms comprise one or more selected from the following: dyspnoea, fever, general malaise, requiring hospital admission for respiratory support.

Preferably, the at least one surrogate marker is the latitude of the individual's residence and vitamin D status is estimated to be low where the latitude of the individual's residence is above 35° North or below 35° South.

Advantageously, the use of surrogate markers to estimate vitamin D status in an individual provides a low cost and readily accessible means of estimating vitamin D status. It is readily acknowledged that at these latitudes, individuals are less likely to be exposed to sufficient UVB radiation to produce the required levels of vitamin D. As dietary vitamin D intake is typically low, individuals' resident at these latitudes are likely to be deficient in vitamin D for at least part of the year.

Optionally, a second surrogate marker is calendar month at the time of carrying out the method and vitamin D status is estimated to be low where the latitude of the individual's residence is above 35° North and the calendar month is between October and April or where the latitude of the individual's residence is below 35° South and the calendar month is between May and September.

Advantageously, taking into account the latitude and season when estimating vitamin D status provides a more accurate estimate of vitamin D status as individuals resident above 35° North or below 35° South are more likely to be vitamin D deficient in the winter months when daylight hours and therefore UVB exposure are reduced.

Optionally, a second surrogate marker is the individual's ethnicity and vitamin D status is estimated to be low where the latitude of the individual's residence is above 35° North and the individual has African, African-Caribbean or South Asian ethnicity or where the latitude of the individual's residence is below 35° South and the individual has African, African-Caribbean or South Asian ethnicity.

Advantageously, taking into account the latitude and ethnicity when estimating vitamin D status provides a more accurate estimate of vitamin D status as individuals with African, African-Caribbean or South Asian ethnicity who are resident above 35° North or below 35° South are more likely to be vitamin D deficient as sunlight, and therefore UVB absorption, through the skin is reduced.

Optionally, the at least one surrogate marker is the individual's BMI and vitamin D status is estimated to be low where the individual has a BMI over 25.

Advantageously, considering BMI when estimating an individual's vitamin D status may provide a more accurate vitamin D status estimate as those with an elevated BMI are more likely to have a low vitamin D status.

Optionally, the at least one surrogate marker is the individual's socioeconomic status and vitamin D status is estimated to be low where the individual has a low socioeconomic status. Advantageously, considering socioeconomic status when estimating an individual's vitamin D status may provide a more accurate vitamin D status estimate as those with a low socioeconomic status are more likely to have a low vitamin D status.

Optionally, socioeconomic status is determined by considering one or more of the following factors: household income, occupation, level of education, place of residence. Socioeconomic status is estimated to be low when one or more of the above factors are considered to be of a lower level or value than the average in the population.

Preferably, vitamin D levels are determined by measuring the level of circulating 25-hydroxyvitamin D and the predetermined threshold is most preferably 75 nmol/L; in some embodiments 50 nmol/L; and in alternative embodiments 20 nmol/L.

Advantageously, levels of circulating 25-hydroxyvitamin D below 75 nmol/L are considered to have negative effects on individuals having at least one of the increased risk haplotypes. Clinically, vitamin D deficiency is typically defined by levels of circulating 25-hydroxyvitamin D below 50 nmol/L. Vitamin D levels are thought to be optimal when levels of circulating 25-hydroxyvitamin D are above 75 nmol/L.

The determination of vitamin D levels is carried out using in vitro techniques.

Alternatively, any suitable in vitro methods can be used to determine vitamin D levels such as 1,25-dihydroxyvitamin D assays.

Optionally, the method further includes the step of determining the gender of the individual.

Optionally, determining when the individual has at least one of the following increased risk HLA haplotypes selected from: DQA1*01:02/DQB1*06:02; DRB1*15:01; DQA1*05:01/DQB1*02:01; and has a vitamin D level below a predetermined threshold, or has a vitamin D status that is estimated to be low based on the at least one surrogate marker; and is male, the individual is at a greater increased risk of developing severe symptoms associated with SARS-CoV-2 infection.

Optionally, the method comprises determining when the individual has at least one of the following increased risk HLA haplotypes selected from: DQA1*01:02/DQB1*06:02; DRB1*15:01; DQA1*05:01/DQB1*02:01; and has a vitamin D level below a predetermined threshold, or has a vitamin D status that is estimated to be low based on the at least one surrogate marker; and the individual has a body mass index (BMI) above 25 the individual is at a greater increased risk of developing severe symptoms associated with SARS-CoV-2 infection.

Optionally, the method includes determining when the individual has at least one of the following increased risk HLA haplotypes selected from: DQA1*01:02/DQB1*06:02; DRB1*15:01; DQA1*05:01/DQB1*02:01; and has a vitamin D level below a predetermined threshold, or has a vitamin D status that is estimated to be low based on the at least one surrogate marker; and the individual is aged above 40 years old, the individual is at a greater increased risk of developing severe symptoms associated with SARS-CoV-2 infection.

Optionally, the method includes determining when the individual has at least one of the following increased risk HLA haplotypes selected from: DQA1*01:02/DQB1*06:02; DRB1*15:01; DQA1*05:01/DQB1*02:01; and has a vitamin D level below a predetermined threshold, or has a vitamin D status that is estimated to be low based on the at least one surrogate marker; and the individual has a low socioeconomic status, the individual is at a greater increased risk of developing severe symptoms associated with SARS-CoV-2 infection.

Optionally, the method further comprises providing individuals having at least one of the increased risk HLA haplotypes and vitamin D levels below a predetermined threshold, or having at least one of the increased risk HLA haplotypes and a vitamin D status which is estimated to be low based on the at least one surrogate marker with a vitamin D supplement.

Advantageously, providing individuals having at least one of the at risk HLA haplotypes and vitamin D levels below a predetermined threshold, or having at least one of the at risk HLA haplotypes and a low estimated vitamin D status with a vitamin D supplement reduces the risk of the individual developing severe symptoms related to SARS-CoV-2 infection.

Optionally, the method further comprises providing individuals having at least one of the increased risk HLA haplotypes and vitamin D levels below a predetermined threshold or individuals having at least one of the increased risk HLA haplotypes and a vitamin D status which is estimated to be low based on the at least one surrogate marker with a vaccination against SARS-CoV-2.

Advantageously, providing a vaccination against the SARS-CoV-2 virus to individuals having at least one of the at risk HLA haplotypes and vitamin D levels below a predetermined threshold, or having at least one of the at risk HLA haplotypes and a low estimated vitamin D status reduces the risk of the individual contracting Covid-19 and developing severe symptoms as a result. Preventing individuals who are most at risk of developing severe symptoms from contracting Covid-19 reduces strain on the healthcare system by reducing the number of hospital admissions.

Optionally, the method further comprises administering individuals having at least one of the increased risk HLA haplotypes and vitamin D levels below a predetermined threshold or individuals having at least one of the increased risk HLA haplotypes and a vitamin D status which is estimated to be low based on the at least one surrogate marker with cod liver oil.

Advantageously, cod liver oil is a source of vitamin D. The administration of cod liver oil to individuals with low vitamin D level or a predicted low vitamin D status and an increased risk HLA haplotype will therefore increase their vitamin D levels and reduce their risk of developing severe symptoms associated with SARS-CoV-2 infection.

Optionally, the step of determining whether the individual has at least one of the increased risk HLA haplotypes further comprises the steps of: obtaining a biological sample from the individual and carrying out sequencing of the HLA genes.

The determination of whether the individual has at least one of the increased risk HLA haplotypes is carried out using in vitro techniques.

Optionally, the method further comprises a step of obtaining a biological sample from the individual and using the biological sample to determine whether the individual has at least one of the increased risk HLA haplotypes.

Optionally, the method further comprises a step of obtaining a biological sample from the individual and using the biological sample to determine the vitamin D level in the individual.

Optionally, the method further comprises a step of using the biological sample to determine the presence SARS-CoV-2.

Advantageously, testing to determine the presence of SARS-CoV-2 allows healthcare professionals to appropriately triage and isolate individuals who are positive for SARS-CoV-2. Where individuals are also determined to be at a high risk of developing severe symptoms associated with SARS-COV-2 infection due to the presence of an increased risk haplotype and low vitamin D levels, these individuals can be carefully monitored and early medical intervention provided to reduce their risk of developing severe symptoms.

Optionally, the biological sample is a biological sample selected from the following: whole blood sample, blood serum sample, blood plasma sample, urine sample, saliva sample, buccal swab.

Optionally, the step of using the biological sample to determine the presence SARS-CoV-2 comprises a molecular test.

According to a fourth aspect of the present invention, there is provided a kit for identifying the risk of an individual developing severe symptoms when infected with SARS-CoV-2, comprising: reagents for determining whether an individual has any of the following increased risk HLA haplotypes selected from: DQA1*01:02/DQB1*06:02; DRB1*15:01; DQA1*05:01/DQB1*02:01; reagents for determining vitamin D status of an individual; and directions for determining the risk of an individual developing severe symptoms when infected with SARS-CoV-2 based on the presence of any of the increased risk HLA haplotypes and the vitamin D status of the individual.

Optionally, the reagents for determining whether an individual has any of the increased risk HLA haplotypes comprises at least one set of oligonucleotide primers.

Advantageously, oligonucleotide primers provide a means of identifying the presence of any of the increased risk HLA haplotypes by standard molecular biological techniques such as PCR.

According to a fifth aspect of the present invention, there is provided a device for identifying the risk of an individual developing severe symptoms when infected with SARS-CoV-2, comprising: a means of inputting a biological sample from the individual; a means of determining whether the sample comprises any of the following increased risk HLA haplotypes selected from: DQA1*01:02/DQB1*06:02; DRB1*15:01; DQA1*05:01/DQB1*02:01; a means of determining the vitamin D levels of the sample or a means of inputting the individual's vitamin D levels or an estimate of the individual's vitamin D status.

Optionally, the biological sample is subject to pre-processing steps before being inputted into the device.

Optionally, the biological sample is a biological sample selected from the following: whole blood sample, blood serum sample, blood plasma sample, urine sample, saliva sample, buccal swab.

Preferably, the device further comprises a means for displaying output data.

Advantageously, a means for displaying output data allows the operator of the device to readily access the data.

Optionally, the means for displaying output data display is a display integrated into the device.

Optionally, the means for displaying output data is an external display such as a computer, mobile phone or tablet device.

Advantageously, this allows the data to be accessed directly from a device such as a clinician's computer.

Optionally, the output data comprises an indication of whether an individual has at least one of the increased risk HLA haplotypes.

Advantageously, this allows an operator to quickly and readily access the data relating to the individual's HLA haplotype. This data can be used to determine the risk of the individual developing severe symptoms if they were to become infected with SARS-CoV-2.

Optionally, the output data indicates an individual's vitamin D status.

Advantageously, this allows an operator to access the data relating to the individual's vitamin D status quickly and readily. This data can be used to determine the risk of the individual developing severe symptoms if they were to become infected with SARS-CoV-2.

Optionally, the output data indicates the risk of an individual developing severe symptoms when infected with SARS-CoV-2.

Advantageously, this provides a quick and readily accessible summary of the risk of an individual developing severe symptoms when infected with SARS-CoV-2 that can be readily interpreted by a lay person.

Optionally, the means of determining whether an individual has at least one of the increased risk HLA haplotypes comprises a molecular HLA assay.

Optionally, the means of determining whether an individual has at least one of the increased risk HLA haplotypes comprises a PCR assay.

Optionally, the means of determining vitamin D level comprises a lateral flow test. Advantageously, a lateral flow test is a fast and cost-effective means of determining vitamin D level in an individual.

Preferably, device is a point of care device.

Advantageously, a point of care device can be used to deliver a result at the point of care and therefore the individual can obtain a fast indication of their risk.

Preferably, the device is a microfluidic device.

Advantageously, microfluidic devices only require a very small sample size meaning that a less invasive sample obtaining method can be used, i.e. a finger prick blood sample. According to a sixth aspect of the present invention, there is provided a method of assessing the risk of an individual developing severe symptoms when infected with SARS-CoV-2, comprising: determining whether the individual has at least one of the following reduced risk HLA haplotypes selected from: DRB1*04:01; DQA1*03:03/DQB1*03:01;HLA-A*03:01; HLA-B*39:01; HLA-C*03:03; DRB*01:01; DQA1*01:01/DQB1*05:01; HLA-B*15:01; DRB1*04:01; HLA-A*03:01; DRB1:04:01; DRB1*08:01; and determining when the individual has at least one of the following reduced risk HLA haplotypes selected from DRB1*04:01; DQA1*03:03/DQB1*03:01;HLA-A*03:01; HLA-B*39:01; HLA-C*03:03; DRB*01:01; DQA1*01:01/DQB1*05:01; HLA-B*15:01; DRB1*04:01; HLA-A*03:01; DRB1:04:01; DRB1*08:01, the individual has a reduced risk of developing severe symptoms associated with SARS-CoV-2 infection.

According to a seventh aspect of the present invention, there is provided a method of assessing the risk of an individual developing severe symptoms when infected with SARS-CoV-2, comprising: determining whether the individual has at least one of the following reduced risk HLA haplotypes selected from: DRB1*04:01; DQA1*03:03/DQB1*03:01;HLA-A*03:01; HLA-B*39:01; HLA-C*03:03; DRB*01:01; DQA1*01:01/DQB1*05:01; HLA-B*15:01; DRB1*04:01; HLA-A*03:01; DRB1:04:01; DRB1*08:01; determining the vitamin D level of the individual, or estimating the vitamin D status of the individual by means of at least one surrogate marker of vitamin D status; and determining when the individual has at least one of the following reduced risk HLA haplotypes selected from: DRB1*04:01; DQA1*03:03/DQB1*03:01; HLA-A*03:01; HLA-B*39:01; HLA-C*03:03; DRB*01:01; DQA1*01:01/DQB1*05:01; HLA-B*15:01; DRB1*04:01; HLA-A*03:01; DRB1:04:01; DRB1*08:01; and has a vitamin D level above a predetermined threshold, or has a vitamin D status that is estimated to be sufficient based on the at least one surrogate marker, the individual has a reduced risk of developing severe symptoms associated with SARS-CoV-2 infection.

Advantageously, identifying the presence of a reduced risk HLA haplotype and sufficient vitamin D levels in an individual indicates that the individual has a reduced risk of developing severe symptoms if infected with SARS-CoV-2. This knowledge can be used when prioritising individuals for receipt of prophylactic treatments such as vaccinations.

Optionally, symptoms comprise one or more selected from: anosmia, headache, fever, general malaise, joint pain, gastrointestinal disturbance, cough and dyspnoea.

Optionally, severe symptoms comprise one or more selected from the following: dyspnoea, fever, general malaise, requiring hospital admission for respiratory support.

Preferably, vitamin D levels are determined by measuring the level of circulating 25-hydroxyvitamin D and the predetermined threshold is most preferably 75 nmol/L; in some alternatives 50 nmol/L; and in other embodiments 20 nmol/L.

Preferably, the at least one surrogate marker is the latitude of the individual's residence and vitamin D status is estimated to be low where the latitude of the individual's residence is above 35° North or below 35° South.

Advantageously, the use of surrogate markers to estimate vitamin D status in an individual provides a low cost and readily accessible means of estimating vitamin D status in an individual. It is readily acknowledged that at these latitudes, individuals are more likely to be exposed to sufficient UVB radiation to produce the required levels of vitamin D.

Optionally, the step of determining whether the individual has at least one of the reduced risk HLA haplotypes further comprises the steps of: obtaining a biological sample from the individual and carrying out sequencing of the HLA genes.

Optionally, the biological sample is a biological sample selected from the following: whole blood sample, blood serum sample, blood plasma sample, urine sample, saliva sample, buccal swab.

According to a further embodiment of the present invention, there is provided a method of stratifying a plurality of individuals based on their risk developing severe symptoms when infected with SARS-CoV-2 by using the method according to the third aspect or the sixth aspect or a combination of the third and sixth aspects of the present invention.

According to a further embodiment of the present invention, there is provided a kit for carrying out the method according to third aspect or the sixth aspect or a combination of the third and sixth aspects.

According to a eighth aspect of the present invention, there is provided a kit for identifying the risk of an individual developing severe symptoms when infected with SARS-CoV-2, comprising: reagents for determining whether an individual has any of the following reduced risk HLA haplotypes selected from: DRB1*04:01; DQA1*03:03/DQB1*03:01; and HLA-A*03:01; reagents for determining vitamin D status of an individual; and directions for determining the risk of an individual developing severe symptoms when infected with SARS-CoV-2 based on the presence of any of the reduced risk HLA haplotypes and the vitamin D status of the individual.

Optionally, the reagents for determining whether an individual has any of the reduced risk HLA haplotypes comprise oligonucleotide primers.

According to a ninth aspect of the present invention, there is provided a device for identifying the risk of an individual developing severe symptoms when infected with SARS-CoV-2, comprising: a means of inputting a biological sample from the individual; a means of determining whether the sample comprises at least one of the following reduced risk HLA haplotypes DRB1*04:01; DQA1*03:03/DQB1*03:01; and HLA-A*03:01; a means of determining the vitamin D status of the sample or a means of inputting the individual's vitamin D status.

Optionally, the biological sample is subject to pre-processing steps before being inputted into the device.

Optionally, the biological sample is a biological sample selected from the following: whole blood sample, blood serum sample, blood plasma sample, urine sample, saliva sample, buccal swab.

Preferably, the device further comprises a means for displaying output data.

Advantageously, a means for displaying output data allows the operator of the device to readily access the data.

Optionally, the means for displaying output data display is a display integrated into the device.

Optionally, the means for displaying output data is an external display such as a computer, mobile phone or tablet device.

Advantageously, this allows the data to be accessed directly from a device such as a clinician's computer.

Optionally, the output data comprises an indication of whether an individual has at least one of the reduced risk HLA haplotypes.

Advantageously, this allows an operator to quickly and readily access the data relating to the individual's HLA haplotype. This data can be used to determine the risk of the individual developing severe symptoms if they were to become infected with SARS-CoV-2.

Optionally, the output data indicates an individual's vitamin D status.

Advantageously, this allows an operator to access the data relating to the individual's vitamin D status quickly and readily. This data can be used to determine the risk of the individual developing severe symptoms if they were to become infected with SARS-CoV-2.

Optionally, the output data indicates the risk of an individual developing severe symptoms when infected with SARS-CoV-2.

Advantageously, this provides a quick and readily accessible summary of the risk of an individual developing severe symptoms when infected with SARS-CoV-2 that can be readily interpreted by a lay person.

Optionally, the means of determining whether an individual has at least one of the reduced risk HLA haplotypes comprises a molecular assay.

Optionally, the means of determining whether an individual has at least one of the reduced risk HLA haplotypes comprises a PCR assay.

Optionally, the means of determining vitamin D level comprises a lateral flow test.

Advantageously, a lateral flow test is a fast and cost-effective means of determining vitamin D level in an individual.

Preferably, device is a point of care device.

Advantageously, a point of care device can be used to deliver a result at the point of care and therefore the individual can obtain a fast indication of their risk.

Preferably, the device is a microfluidic device.

Advantageously, microfluidic devices only require a very small sample size meaning that a less invasive sample obtaining method can be used, i.e. a finger prick blood sample.

According to a tenth aspect of the present invention, there is provided method of assessing the risk of an individual developing severe symptoms when infected with SARS-CoV-2, the method comprising: determining whether the individual has at least one HLA haplotype associated with an increased risk of developing severe symptoms associated with SARS-CoV-2 infection; administrating the subject with a prophylactic or therapeutic to prevent or ameliorate the onset of severe symptoms associated with SARS-CoV-2 infection.

Optionally, the HLA haplotypes associated with a high risk of exhibiting severe symptoms associated with SARS-CoV-2 infection are selected from the following: DRB1*04:01; DQA1*03:03/DQB1*03:01; HLA-A*03:01; HLA-B*39:01; HLA-C*03:03; DRB*01:01; DQA1*01:01/DQB1*05:01; HLA-B*15:01; DRB1*04:01; HLA-A*03:01; DRB1:04:01; DRB1*08:01;

Optionally, the method may be implemented by a computer program.

Optionally, the HLA haplotypes associated with a high risk of exhibiting severe symptoms associated with SARS-CoV-2 infection are identified by a machine-learning algorithm.

Optionally, the machine-learning algorithm is trained using the HLA haplotypes and symptom severity of patients with known SARS-CoV-2 infection.

According to an eleventh aspect of the present invention, there is provided a method of assessing the risk of an individual developing severe symptoms when infected with SARS-CoV-2, the method comprising: determining whether the individual has at least one HLA haplotype associated with an increased risk of developing severe symptoms when infected with SARS-CoV-2 according to the first, second, or third aspect of the present invention; determining whether the individual has at least one HLA haplotype associated with a reduced risk of developing severe symptoms when infected with SARS-CoV-2 according to the sixth or seventh aspects of the present invention; ascribing a risk value to the increased risk HLA haplotypes and/or reduced risk HLA haplotypes; combining the risk values to determine an overall risk value associated with developing severe symptoms when infected with SARS-CoV-2; wherein a high risk value corresponds to an increased risk of developing severe symptoms when infected with SARS-CoV-2 and a low risk value corresponds to a reduced risk of developing severe symptoms when infected with SARS-CoV-2.

Optionally, the method further comprises a step of determining at least one of the additional risk factors from the following: the age of the individual; the gender of the individual; the BMI of the individual; ascribing a risk value to the at least one additional risk factor; combining the risk values of the additional risk factors with the risk values of the HLA haplotypes to determine an overall risk value associated with developing severe symptoms when infected with SARS-CoV-2.

Optionally, the method is carried out by a computer program.

Various further features and aspects of the invention are defined in the claims.

Unless defined otherwise, all technical and scientific terms used herein have the meaning commonly understood by a person skilled in the art to which this invention belongs.

To assist the reader, the following terms have the meanings ascribed to them below, unless specified otherwise.

The term “determining” refers to the collection of data and/or information from any source. For example, the data and/or information can be collected by primary means, i.e. by carrying out an assay on a biological sample from an individual to determine the HLA haplotype. The data and/or information can also be collected from secondary sources, i.e. by retrieving the HLA haplotype from the individual's medical records.

The term “body mass index” or “BMI” refers to a value derived from the mass and height of an individual. BMI can be calculated by dividing the weight of an individual in kilograms divided by their height in metres squared (BMI=mass(kg)/(height(meters)²)). The skilled person would understand that any suitable alternative measure could be used to indicate an individual's weight normalised according to their height.

The nomenclature of the HLA system used throughout the present specification is that formally established by the World Health Organization HLA Nomenclature Committee

### DETAILED DESCRIPTION

The inventors of the present invention sought to better understand the factors which contributed to the likelihood of an individual developing severe symptoms as a result of SARS-CoV-2 infection. Specifically, the inventors sought to establish whether there was a correlation between HLA haplotype and severity of symptoms in individuals with Covid-19.

HLA haplotypes have been identified as factors modulating disease severity in a number of conditions affecting the immune system such as Multiple Sclerosis and Diabetes.

### Study on HLA Haplotype and Covid-19 Symptom Severity

Staff and patients were recruited from 2 teaching hospitals in the North East of England. They formed four different groups based on symptoms/absence of symptoms.
1. Hospital staff who tested positive for COVID antibodies on screening blood tests or COVID antigen on screening (swabs). They were completely asymptomatic, unaware they had been infected with COVID
2. Hospital staff who tested positive for COVID antibodies on screening blood tests. They reported local symptoms only (i.e. loss of smell/taste)
3. Hospital staff who tested positive for COVID antibodies and had systemic symptoms but did not require hospital admission
4. Patients who were previously well, no significant comorbidities who required admission for oxygen or more advanced respiratory support following infection with COVID.

Participants gave saliva samples for next generation sequencing of HLA class I and II genes.

### Statistical Analysis Step 1

Alleleic frequencies of groups 1 and 4 were compared to each other and then to a control group of 250 patients with degenerative osteoarthritis taken from the same area of England (no known link to specific HLA types).

A check against a larger national group was carried to confirm that the local osteoarthritis group showed no deviation from a larger national sample. This was confirmed. Dosage estimates from 8514 National Blood Service and 1958 Birth Cohort controls were also analysed by logistic regression. These controls had passed previously derived quality control checks and were genotyped at 143,006 SNPs, of which 7848 fell within the extended MHC region on chromosome 6 (ranging from 25,650,000 to 33,426,000 base pairs, Build). Imputation of classical HLA alleles was implemented with HIBAG(34) for HLA-A, HLA-B, HLA-C, HLA-DPB1, HLA-DQA1, HLA-DQB1 and HLA-DRB1. HIBAG produces a posterior probability for each possible genotype (i.e. for each combination of two alleles, including combinations that have lower probabilities than the best combination). This genotype-based output enables averaging over the possible genotype combinations (while allowing appropriately for imputation uncertainty) in order to obtain dosage estimates for each allele. Adjustment was made for age and sex.

Two independent reviews were made by statisticians with expertise in statistical genetic analysis.

### Step 2: Binary Logistic Regression

The genes identified in step 1 were entered into a regression model to determine how well the model predicted severe versus asymptomatic COVID infection.

### Results 1: Comparison of Alleleic Frequencies Between Groups

A. Asymptomatics Versus Controls

| Allele | Odds ratio | Standard error | P value |
|---|---|---|---|
| B39.01 | 4.482 | 0.743 | 0.044 |
| C03.03 | 2.662 | 0.440 | 0.026 |
| C04.01 | 0.143 | 0.832 | 0.019 |
| DQA01.01 | 0.133 | 0.764 | 0.008 |
| DQB05.01 | 0.197 | 0.642 | 0.011 |
| DRB1_01.01 | 0.180 | 0.775 | 0.027 |

Patients infected with COVID are more likely to remain asymptomatic compared to the background population if they have:
B*39:01
C*03:03

Patients infected with COVID who remained asymptomatic are less likely to have the haplotype:
DRB1*01:01
DQA1*01:01
DQB1*05:01

B. Severe Versus Asymptomatic

| Allele | OR | SE | P value |
|---|---|---|---|
| A*03:01 | 0.607 | | 0.079 |
| A*11:01 | 4.688 | | 0.027 |
| B15.01 | 0.170 | 0.753 | 0.018 |
| B44.03 | 5.516 | 0.706 | 0.016 |
| C04.01 | 7.533 | 0.988 | 0.041 |
| DQB1*06:02 (linked to DRB1*15) | | | 0.064 |
| DRB1_01:01 | 10 | | 0.039 |
| DRB1_04.01 | 0.181 | 0.568 | 0.003 |
| DRB1_08:01 | 0 | | 0.043 |
| DRB1*15:01 | 2.666 | | 0.094 |

Patients infected with COVID who have:
B*44:03
C*04:01

Are more likely to develop severe disease

Patients infected with COVID who have:
B*15:01
DRB1*04:01 (linked to DQA1*03:03/DQB1*03:01)

Are less likely to develop severe disease

Linked haplotypes:

| Haplotype | | | | | | | | | |
|---|---|---|---|---|---|---|---|---|---|
| DRB1/DQA1/ | | | | | | | | | |
| DQB1 | Case | Control | Ca-Freq | Co-Freq | Odds-R | 95% Lo | 95% Hi | Chisq | P-value |
| 101-101-501 | 7 | 2 | 0.07447 | 0.01695 | 1 | 1 | 1 | 4.258 | 0.03906 |
| 401-301-302 | 2 | 9 | 0.02128 | 0.07627 | 0.06349 | 0.007072 | 0.5701 | 3.217 | 0.07289 |
| 401-303-301 | 2 | 12 | 0.02128 | 0.1017 | 0.04762 | 0.005435 | 0.4172 | 5.486 | 0.01917 |
| 801-401-402 | 0 | 5 | 0 | 0.04237 | 0 | 0 | 0 | 4.079 | 0.04341 |
| 1501-102-602 | 21 | 15 | 0.2234 | 0.1271 | 0.4 | 0.07267 | 2.202 | 3.441 | 0.06361 |

C. Severe Versus Controls

| Allele | OR | SE | P value |
|---|---|---|---|
| A03.01 | 0.366 | 0.412 | 0.015 |
| DRB1_04.05 | 12.16 | 1.174 | 0.033 |
| DRB1_04.01 | 0.329 | 0.538 | 0.039 |
| DRB1_04.03 | 4.119 | 0.838 | 0.091 |

Alleles linked to increased risk of severe disease compared to background population
DRB1*04:03
DRB1*04:05

Alleles linked to reduced risk of severe disease compared to background population:
A*03:01
DRB1*04:01

### Results 2: Binary Logistic Regression

Inclusion of asymptomatic versus severe patients only

R squared value=78% p<0.001

| from\to | 0 | 1 | Total | % correct |
|---|---|---|---|---|
| 0 | 50 | 9 | 59 | 84.75% |
| 1 | 8 | 39 | 47 | 82.98% |
| Total | 58 | 48 | 106 | 83.96% |

| | | | | | Wald | Wald |
|---|---|---|---|---|---|---|
| | | | Wald | | Lower | Upper |
| | Risk | Standard | Chi- | Pr > | bound | bound |
| Source | Value | error | Square | Chi² | (95%) | (95%) |
| Age | 0.627 | 0.178 | 12.446 | 0.000 | 0.278 | 0.975 |
| BMI | 0.331 | 0.159 | 4.322 | 0.038 | 0.019 | 0.643 |
| Sex-F | 0.000 | 0.000 | | | | |
| Sex-M | 0.350 | 0.154 | 5.158 | 0.023 | 0.048 | 0.653 |
| B*15:01-0 | 0.000 | 0.000 | | | | |
| B*15:01-1 | −0.198 | 0.179 | 1.220 | 0.269 | −0.548 | 0.153 |
| B*39:01-0 | 0.000 | 0.000 | | | | |
| B*39:01-1 | −0.307 | 0.228 | 1.816 | 0.178 | −0.754 | 0.140 |
| B*44:03-0 | 0.000 | 0.000 | | | | |
| B*44:03-1 | 0.290 | 0.158 | 3.367 | 0.067 | −0.020 | 0.600 |
| HLA-A*03-0 | 0.000 | 0.000 | | | | |
| HLA-A*03-1 | −0.288 | 0.149 | 3.736 | 0.053 | −0.579 | 0.004 |
| HLA-A*11-0 | 0.000 | 0.000 | | | | |
| HLA-A*11-1 | 0.185 | 0.151 | 1.503 | 0.220 | −0.111 | 0.480 |
| DRB1*04:01-0 | 0.000 | 0.000 | | | | |
| DRB1*04:01-1 | −0.211 | 0.168 | 1.580 | 0.209 | −0.540 | 0.118 |
| DRB1*08-0 | 0.000 | 0.000 | | | | |
| DRB1*08-1 | −0.199 | 0.193 | 1.068 | 0.301 | −0.577 | 0.179 |
| DRB1*04:05 | 0.000 | 0.000 | | | | |
| or 04:03-0 | | | | | | |
| DRB1*04:05 | 0.087 | 0.157 | 0.310 | 0.578 | −0.220 | 0.394 |
| or 04:03-1 | | | | | | |
| DRB1*15-0 | 0.000 | 0.000 | | | | |
| DRB1*15-1 | 0.137 | 0.151 | 0.824 | 0.364 | −0.159 | 0.433 |

The above table shows indicative risk values that could be attributed to the haplotypes and characteristics such as age, BMI and gender as part of a risk assessment algorithm. It would be understood that the above data set could be further trained and optimised using larger volumes of patient data and the risk values ascribed optimised accordingly.

Additional analysis identified that the HLA haplotype DRB1*04:02 is also linked to an increased risk of developing severe symptoms associated with SARS-CoV-2 and that a risk value can be ascribed to this haplotype in a similar manner to the above.

### Additional Study on HLA Haplotype and Covid-19 Symptom Severity

### Experimental Methods

A group of 132 individuals who had received a positive SARS-COV-2 antigen test were identified. The individuals were asked to report the symptoms they had experienced as a result of the SARS-CoV-2 infection. Symptoms were stratified into 4 groups (group 1 to 4), group 1 represents individuals who were asymptomatic, group 2 contains individuals who experienced local symptoms such as anosmia, group 3 represents individuals who experienced systemic symptoms such as fever and flu-like symptoms, and group 4 contains those requiring admission to hospital.

Additional data was collected from the individuals such as height, weight, gender and age. Biological samples were obtained from each of the individuals and these biological samples were used for HLA haplotyping.

To determine the HLA haplotypes, DNA was extracted from buccal swab and sputum samples using the Roche MagnaPure Compact automated platform (Roche Holding AG, Switzerland). DNA was the quantified using the Thermo Fisher Qubit dsDNA BR Assay kit and standardised to 25 ng/ul.

HLA genotyping was performed using the One Lambda AllType NGS kits (One Lambda, USA), with the Illumina MiSeq platform (Illumina, USA). Full gene sequencing was carried out for HLA-A, -B, -C, -DQA1 and -DPA1 and partial gene sequencing (omission of exon 1) for HLA-DRB1, -DRB345, -DQB1 and -DPB1. HLA genotypes were analysed using One Lambda TypeStream Visual 1.3 software (One Lambda, USA).

Control data was obtained from two sources. One source was a group of patients with degenerative osteoarthritis, this group was used as no link was predicted between degenerative osteoarthritis and Covid-19 symptom severity and the patients were located in North East England as were the Covid-19 study individuals. The HLA haplotypes of the degenerative osteoarthritis patients were known from a previous study. The second control group used was a general background population based in North East England. Both control groups were combined for use as background population group with which to compare the Covid-19 group.

Data relating to the binding of SARS-Cov-2 to HLA molecules resulting from different haplotypes (as shown in tables 2 and 3) was obtained using validated software (NetMHCIIpan-4.0 server) to predict peptide binding to any MHC II molecule of known sequence using Artificial Neural Networks (ANNs). The software is trained on an extensive dataset of over 500,000 measurements of Binding Affinity (BA) and Eluted Ligand mass spectrometry (EL), covering the three human MHC class II isotypes HLA-DR, HLA-DQ, HLA-DP, as well as the mouse molecules (H-2). The MHC II protein sequences were entered in FASTA format.

The output of the model is a prediction score for the likelihood of a peptide to be naturally presented by and MHC II receptor of choice. The output also includes a percentage rank score, which normalizes the prediction score by comparing to the prediction of a set of random peptides. Optionally, the model also outputs BA prediction and percentage rank scores.

### Results

It is clear from the data that certain haplotypes are more prevalent in the asymptomatic (group 1) individuals or the severe (group 4) individuals, indicating that correlations exist between HLA haplotype and likelihood of developing a severe disease as a result of SARS-CoV-2 infection.

Surprisingly, the inventors discovered that one particular HLA haplotype (DQA1*01:02/DQB1*06:02) appears at a higher rate in both the severe and asymptomatic individuals than in the background population. When compared to the background population, the rate at which this haplotype appears in the severe individuals is significantly higher (p<0.001).

The HLA-DQ protein arising from the DQA1*01:02/DQB1*06:02 haplotype is predicted to bind strongly to the SARS-CoV-2 virus (table 2).

A strong interaction between the SARS-CoV-2 virus and HLA-DQ protein is likely to be protective from severe disease as a result of an increased antibody response. Given that this haplotype appears with a higher frequency in both the severe and asymptomatic individuals, this finding is surprising. The inventors sought to investigate why this particular haplotype is prevalent in the severe individuals given that it is predicted to strongly bind to the SARS-CoV-2 virus.

There is strong linkage between the HLA-DQ and HLA-DR genes meaning many specific haplotypes are commonly inherited together. The DQA1*01:02/DQB1*06:02 haplotype also shows strong linkage with DRB1*15:01 which is also more prevalent in the severely affected individuals (Table 1).

The inventors found that the DQA1*01:02/DQB1*06:02 haplotype is modulated by vitamin D. Individuals having low vitamin D levels and the DQA1*01:02/DQB1*06:02 haplotype are at a higher risk of having severe symptoms resulting from SARS-CoV-2 infection than those with adequate or high vitamin D levels and the DQA1*01:02/DQB1*06:02 haplotype.

TABLE 1

| | Frequency in severe individuals v |
|---|---|
| HLA haplotype | background population (p-value) |
| DQA1*01:02/DQB1*06:02 | <0.001 |
| DRB1*15:01 | <0.001 |

Additional analysis also identified DQA1*05:01/DQB1*02:01 haplotype as being more prevalent in severely affected individuals. The protein resulting from this haplotype is also vitamin D responsive. As is the case with the DQA1*01:02/DQB1*06:02 haplotype, the protein resulting from the DQA1*05:01/DQB1*02:01 haplotype is predicted to bind strongly to the SARS-CoV-2 virus (Table 2).

TABLE 2

| | | Highest | | |
|---|---|---|---|---|
| | | Ranking | | |
| | | SARS-COV-2 | | |
| | | Epitope | | Change in |
| | | Predicted | Epitope | Haplotype |
| | | to be Bound | rank EL | Frequency |
| | | by the | (percen- | with |
| DQA1/DQB1 | DRB1 | HLA-DQ | tile | Increasing |
| Haplotype | Linkage | Protein | rank) | Latitude |
| DQA1*01:02/ | DRB1*15:01 | VTQQLIRAAEI | 0 | Increase |
| DQB1*06:02 | | RASA | | |
| DQA1*05:01/ | DRB1*03:01 | GRDIADTTDAV | 0.71 | Increase |
| DQB1*02:01 | | RDPQ | | |

Interestingly, the inventors observed that the increased risk HLA haplotypes appeared to be positively correlated with increased latitude. Therefore, demonstrating that the increased risk haplotypes are more frequent at higher latitudes where vitamin D levels are more likely to be low.

The inventors have further identified that the risk of developing severe symptoms in individuals having these increased risk haplotypes can be further increased depending on the gender of the individual. Individuals with the following haplotypes are at a higher risk of developing severe symptoms if they are male: DQA1*01:02/DQB1*06:02; DRB1*15:01; DQA1*05:01/DQB1*02:01.

The inventors have also identified that the DRB1*04:01 haplotype appears less frequently in the severely affected individuals than the general population (p=0.004), and that it too is vitamin D responsive. This haplotype is therefore indicative of a reduced risk of developing severe symptoms as a result of SARS-CoV-2 infection. The DRB1*04:01 haplotype is often linked to the DQA1*03:03/DQB1*03:01 haplotype which also appears at a lower frequency in the severe patients than the general population.

Proteins resulting from the HLA haplotypes DRB1*04:01 and DQA1*03:03/DQB1*03:01 are predicted to bind strongly to the SARS-CoV-2 virus (table 3). The frequency of many of these haplotypes are also positively correlated with increasing latitude.

TABLE 3

| | | SARS-COV-2 | | Change in |
|---|---|---|---|---|
| | | Epitope | | Haplotype |
| | | Predicted | | Frequency |
| | | to be Bound | Epitope | with |
| | | by HLA-DQ | rank EL | Increasing |
| DQA1/DQB1 Haplotype | DRB1 | Protein | (percentile) | Latitude |
| DQA1*03:01/DQB1*03:01 | Linkage | TQQLIRAAEIR | 0.22 | Increase |
| | | ASAN | | |
| DQA1*03:01/DQB1*03:02 | DRB1*04:01 | LNRALTGIAVE | 0.04 | Increase |
| | | QDKN | | |
| DQA1*03:03/DQB1*03:01 | DRB1*04:01 | TQQLIRAAEIR | 0.22 | Increase |
| | | ASAN | | |
| DQA1*03:03/DQB1*03:01 | DRB1*04:07 | TQQLIRAAEIR | 0.22 | None |
| | | ASAN | | |
| DQA1*03:03/DQB1*03:01 | DRB1*04:08 | TQQLIRAAEIR | 0.22 | Increase |
| | | ASAN | | |

### EXAMPLE 1

### Laboratory Testing Method

A buccal swab and saliva sample are collected from an individual for determining the HLA haplotype. DNA is extracted from the buccal swab and sputum sample using the Roche MagnaPure Compact automated platform (Roche Holding AG, Switzerland). DNA is quantified using the Thermo Fisher Qubit dsDNA BR Assay kit and standardised to 25 ng/ul.

HLA genotyping is performed using the One Lambda AllType NGS kits (One Lambda, USA), with the Illumina MiSeq platform (Illumina, USA). Full gene sequencing is carried out for HLA-A, -B, -C, -DQA1 and -DPA1 and partial gene sequencing (omission of exon 1) for HLA-DRB1, -DRB345, -DQB1 and -DPB1. HLA genotypes are analysed using One Lambda TypeStream Visual 1.3 software (One Lambda, USA).

The haplotypes are manually checked to determine whether the individual has any of the increased risk haplotypes (DQA1*01:02/DQB1*06:02; DRB1*15:01; DQA1*05:01/DQB1*02:01).

A blood sample was also collected from the same individual for determining the vitamin D levels. 25-hydroxyvitamin D [25(OH)D] was measured using the Roche immunoassay C802, and measured in nmol per litre.

The 25(OH)D levels are manually reviewed to determine whether they are lower than the predetermined threshold of 75 nmol/L.

If the individual has an at risk haplotype and the vitamin D level is below the predetermined threshold, the individual has an increased risk of developing severe symptoms associated with SARS-CoV-2 infection. The individual may be supplied with supplements to increase their vitamin D levels (vitamin D supplements or cod liver oil) and/or be administered with a vaccination against SARS-CoV-2 virus.

### EXAMPLE 2

### Laboratory Testing Method With Use of a Surrogate Marker of Vitamin D Status

A buccal swab and saliva sample are collected from an individual for determining the HLA haplotype. HLA haplotyping and determination of the presence of increased risk haplotypes is carried out as described in example 1.

Information relating to the individual's geographical residence is gathered. If the individual resides at a location having a latitude of above 35° North or below 35° South, the individual's vitamin D status is estimated to be low.

If the individual has an at risk haplotype and the vitamin D status is estimated to be low, the individual has an increased risk of developing severe symptoms associated with SARS-CoV-2 infection. The individual may be supplied with supplements to increase their vitamin D levels (vitamin D supplements or cod liver oil) and/or be administered with a vaccination against SARS-CoV-2 virus.

### EXAMPLE 3

### Algorithm Using Secondary HLA Haplotype Data With Use of a Surrogate Marker of Vitamin D Status

An individual's HLA haplotype is determined by obtaining the data from their medical records. Certain individuals may have had HLA haplotyping as part of previous medical investigations and therefore their HLA haplotypes are known and can be obtained from their medical records. The presence of an increased risk HLA haplotype can be determined by comparison with the following list of at risk HLA haplotypes: DQA1*01:02/DQB1*06:02; DRB1*15:01; DQA1*05:01/DQB1*02:01.

The individual's vitamin D status is determined by the use of a surrogate marker as described above in example 2.

If the individual has an at risk haplotype and the vitamin D status is estimated to be low, the individual has an increased risk of developing severe symptoms associated with SARS-CoV-2 infection. The determination of an individual's risk may be carried out manually by a person comparing their HLA haplotype and estimated vitamin D status with threshold values, alternatively this may be carried out by a computer program. The individual may be supplied with supplements to increase their vitamin D levels (vitamin D supplements or cod liver oil) and/or be administered with a vaccination against SARS-CoV-2 virus.

The above embodiments are examples of how methods according to the present invention may be carried out. The skilled person would understand that any suitable alternative methodologies could be used in the determination of vitamin D level or estimating vitamin D status and HLA haplotyping.

All of the features disclosed in this specification (including any accompanying claims, abstract and drawings), and/or all of the steps of any method or process so disclosed, may be combined in any combination, except combinations where at least some of such features and/or steps are mutually exclusive. Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features. The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims are generally intended as “open” terms (e.g., the term “including” or “comprising” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

It will be appreciated that various embodiments of the present disclosure have been described herein for purposes of illustration, and that various modifications may be made without departing from the scope of the present disclosure. Accordingly, the various embodiments disclosed herein are not intended to be limiting, with the true scope being indicated by the following claims.

1. A method of assessing the risk of an individual developing severe symptoms when infected with SARS-CoV-2, comprising:
determining whether the individual has at least one of the following reduced risk HLA haplotypes selected from: DRB1*04:01; DQA1*03:03/DQB1*03:01; HLA-A*03:01; HLA-B*39:01; HLA-C*03:03; DRB*01:01; DQA1*01:01/DQB1*05:01; HLA-B*15:01; HLA-A*03:01; DRB1*08:01;
and determining when the individual has at least one of the following reduced risk HLA haplotypes selected from: DRB1*04:01; DQA1*03:03/DQB1*03:01; HLA-A*03:01; HLA-B*39:01; HLA-C*03:03; DRB*01:01; DQA1*01:01/DQB1*05:01; HLA-B*15:01; HLA-A*03:01; DRB1*08:01;
the individual has a reduced risk of developing severe symptoms associated with SARS-CoV-2 infection.

2. The method according to claim 1 comprising:
determining whether the individual has at least one of the following reduced risk HLA haplotypes selected from: DRB1*04:01; DQA1*03:03/DQB1*03:01; and HLA-A*03:01;
determining the vitamin D level of the individual, or estimating the vitamin D status of the individual by means of at least one surrogate marker of vitamin D status;
and determining when the individual has at least one of the following reduced risk HLA haplotypes selected from: DRB1*04:01; DQA1*03:03/DQB1*03:01; and HLA-A*03:01;
and has a vitamin D level above a predetermined threshold, or has a vitamin D status that is estimated to be sufficient based on the at least one surrogate marker, the individual has a reduced risk of developing severe symptoms associated with SARS-CoV-2 infection.

3. The method according to claim 2, wherein vitamin D levels are determined by measuring the level of circulating 25-hydroxyvitamin D and the predetermined threshold is 75 nmol/L; 50 nmol/L; or 20 nmol/L.

4. The method according to claim 2, wherein the at least one surrogate marker is the latitude of the individual's residence and vitamin D status is estimated to be low where the latitude of the individual's residence is above 35° or below 35°.

5. The method according to claim 2, wherein the method further comprises a step of obtaining a biological sample from the individual and using the biological sample to determine whether the individual has at least one of the reduced risk HLA haplotypes and/or to determine the vitamin D level in the individual and/or to determine the presence of SARS-CoV-2.

6. A kit for identifying the risk of an individual developing severe symptoms when infected with SARS-CoV-2, comprising:
reagents for determining whether an individual has any of the following reduced risk HLA haplotypes selected from: DRB1*04:01; DQA1*03:03/DQB1*03:01; and HLA-A*03:01;
reagents for determining vitamin D status of an individual;
and directions for determining the risk of an individual developing severe symptoms when infected with SARS-CoV-2 based on the presence of any of the reduced risk HLA haplotypes and the vitamin D status of the individual.

7. The kit according to claim 6, wherein the reagents for determining whether an individual has any of the reduced risk HLA haplotypes comprise oligonucleotide primers.

8. A device for identifying the risk of an individual developing severe symptoms when infected with SARS-CoV-2, comprising:
a means of inputting a biological sample from the individual;
a means of determining whether the sample comprises one or more of the reduced risk HLA haplotypes selected from the following: DRB1*04:01; DQA1*03:03/DQB1*03:01; and HLA-A*03:01;
a means of determining the vitamin D status of the sample or a means of inputting the individual's vitamin D status.

9. The device according to claim 8, wherein the output data comprises an indication of at least one of the following: whether an individual has at least one of the reduced risk HLA haplotypes; an individual's vitamin D status; the risk of an individual developing severe symptoms when infected with SARS-CoV-2.

10. A method of assessing the risk of an individual developing severe symptoms when infected with SARS-CoV-2, the method comprising:
determining whether the individual has at least one of the following increased risk HLA haplotypes selected from DQA1*01:02/DQB1*06:02; DRB1*15:01; DQA1*05:01/DQB1*02:01; HLA-B*44:03; HLA-C*04:01; HLA-A*11:01; DRB1*04:03; DRB1*04:05; DRB1*04:02;
and when the individual has at least one of the following increased risk HLA haplotypes selected from: DQA1*01:02/DQB1*06:02; DRB1*15:01; DQA1*05:01/DQB1*02:01; HLA-B*44.03; HLA-C*04:01; HLA-A*11:01; DRB1*04:03; DRB1*04:05; DRB1*04:02;
determining that the individual is at increased risk of developing severe symptoms associated with SARS-CoV-2 infection.

11. The method according to claim 10, comprising the further steps:
determining the vitamin D level of the individual, or estimating the vitamin D status of the individual by means of at least one surrogate marker of vitamin D status;
and where the individual with at least one of the increased risk HLA haplotypes selected from: DQA1*01:02/DQB1*06:02; DRB1*15:01; DQA1*05:01/DQB1*02:01;
and also has a vitamin D level below a predetermined threshold, or has a vitamin D status that is estimated to be low based on the at least one surrogate marker, determining that the individual is at increased risk of developing severe symptoms associated with SARS-CoV-2 infection.

12. The method according to claim 10, the method comprising:
wherein the step of determining whether the individual has at least one of the following increased risk HLA haplotypes selected from DQA1*01:02/DQB1*06:02; DRB1*15:01; DQA1*05:01/DQB1*02:01; HLA-B*44:03; HLA-C*04:01; HLA-A*11:01; DRB1*04:03; DRB1*04:05; DRB1*04:02, comprises determining whether the individual has at least one of the following increased risk HLA haplotypes selected from: DQA1*01:02/DQB1*06:02; DRB1*15:01; DQA1*05:01/DQB1*02:01.

13. The method according to claim 10, further comprising the step of determining at least one of the following: individual's age; individual's BMI; individual's gender;
wherein it is determined that the individual is at a greater increased risk of developing severe symptoms associated with SARS-CoV-2 infection if it is determined that the individual has one or more of an age of 40 years or more; a BMI over 25; a biological sex determination of male.

14. The method according to claim 11, wherein the at least one surrogate marker is the latitude of the individual's residence and vitamin D status is estimated to be low where the latitude of the individual's residence is above 35° North or below 35° South.

15. The method according to claim 11, wherein a second surrogate marker is the individual's ethnicity and vitamin D status is estimated to be low where the latitude of the individual's residence is above 35° North and the individual has African, African-Caribbean or South Asian ethnicity or where the latitude of the individual's residence is below 35° South and the individual has African, African-Caribbean or South Asian ethnicity.

16. (canceled)

17. (canceled)

18. (canceled)

19. (canceled)

20. A kit for identifying the risk of an individual developing severe symptoms when infected with SARS-CoV-2, comprising:
reagents for determining whether an individual has any of the following increased risk HLA haplotypes selected from: DQA1*01:02/DQB1*06:02, DRB1*15:01; DQA1*05:01/DQB1*02:01;
reagents for determining vitamin D status of an individual;
and directions for determining the risk of an individual developing severe symptoms when infected with SARS-CoV-2 based on the presence of any of the increased risk HLA haplotypes and the vitamin D status of the individual.

21. A kit according to claim 20, wherein the reagents for determining whether an individual has any of the increased risk HLA haplotypes comprises at least one set of oligonucleotide primers.

22. A device for identifying the risk of an individual developing severe symptoms when infected with SARS-CoV-2, comprising:
a means of inputting a biological sample from the individual;
a means of determining whether the sample comprises any of the following increased risk HLA haplotypes selected from: DQA1*01:02/DQB1*06:02; DRB1*15:01 DQA1*05:01/DQB1*02:01; HLA-B*44:03; HLA-C*04: 01; HLA-A*11:01; DRB1*04:03; DRB1*04:05; DRB1*04:02.

23. The device according to claim 22 comprising:
a means of inputting a biological sample from the individual;
a means of determining whether the sample comprises any of the following increased risk HLA haplotypes selected from: DQA1*01:02/DQB1*06:02; DRB1*15:01 DQA1*05:01/DQB1*02:01;
a means of determining the vitamin D levels of the sample or a means of inputting the individual's vitamin D levels or an estimate of the individual's vitamin D status.

24. The device according to claim 22, comprising a means for providing output data wherein the output data comprises an indication of at least one of the following: whether an individual has at least one of the increased risk HLA haplotypes; an individual's vitamin D status; the risk of an individual developing severe symptoms when infected with SARS-COV-2.

25. A method of stratifying a plurality of individuals based on their risk of developing severe symptoms when infected with SARS-CoV-2 comprising using the method according to claim 1.

26. (canceled)

27. (canceled)

28. A method of assessing the risk of an individual developing severe symptoms when infected with SARS-CoV-2, the method comprising:
determining whether the individual has at least one HLA haplotype associated with an increased risk of developing severe symptoms associated with SARS-CoV-2 infection;
administrating the subject with a prophylactic or therapeutic to prevent or ameliorate the onset of severe symptoms associated with SARS-CoV-2 infection.

29. A method of assessing the risk of an individual developing severe symptoms when infected with SARS-CoV-2, the method comprising:
determining whether the individual has at least one HLA haplotype associated with a reduced risk of developing severe symptoms when infected with SARS-CoV-2 selected from: DRB1*04:01; DQA1*03:03/DQB1*03:01; HLA-A*03:01; HLA-B*39:01; HLA-C*01:03; DRB*01:01; DQA1*01:01/DQB1*05:01; HLA-B*15:01; HLA-A*03:01; DRB1*08:01;
determining whether the individual has at least one HLA haplotype associated with an increased risk of developing severe symptoms when infected with SARS-CoV-2 selected from DQA1*01:02/DQB1*06:02; DRB1*15:01; DQA1*05:01/DQB1*02:01; HLA-B*44:03; HLA-C*04:01; HLA-A*11:01; DRB1*04:03; DRB1*04:05; DRB1*04:02;
ascribing a risk value to the reduced risk HLA haplotypes and/or increased risk HLA haplotypes;
combining the risk values to determine an overall risk value associated with developing severe symptoms when infected with SARS-CoV-2; wherein a low risk value corresponds to a reduced risk of developing severe symptoms when infected with SARS-CoV-2 and a high risk value corresponds to an increased risk of developing severe symptoms when infected with SARS-CoV-2.

30. (canceled)

31. A method of stratifying a plurality of individuals based on their risk of developing severe symptoms when infected with SARS-CoV-2 comprising using the method according to claim 10.
